(19) United States
(12) Patent Application Publication
(10) Pub. No.: US 2020/0060536 A1
(43) Pub. Date: Feb. 27, 2020

(54) DEVICES, SYSTEMS AND METHODS FOR IN VIVO CLEANING OF MEDICAL INSTRUMENT SURFACE

(71) Applicants: BOARD OF REGENTS, THE UNIVERSITY OF TEXAS SYSTEM, Austin, TX (US); SETON HEALTHCARE FAMILY, Austin, TX (US)

(72) Inventors: Christopher RYLANDER, Austin, TX (US); Christopher IDELSON, Austin, TX (US); William MEADOR, Austin, TX (US); John UECKER, Austin, TX (US); Jawad ALI, Austin, TX (US)

(21) Appl. No.: 16/461,280

(22) PCT Filed: Nov. 15, 2017
(86) PCT No.: PCT/US2017/061676

§ 371 Date: May 15, 2019

Related U.S. Application Data
(60) Provisional application No. 62/422,785, filed on Nov. 16, 2016.

(51) Int. Cl. A61B 1/12; A61B 1/313

(52) U.S. Cl. CPC A61B 1/126; A61B 1/3132

(57) ABSTRACT

Devices, systems and methods for cleaning a surface of a medical instrument in vivo. In particular embodiments of the device, a longitudinal member extends along a shaft of a laparoscope and a transverse member removes matter from a lens of the laparoscope.

### CROSS-REFERENCE TO RELATED APPLICATIONS

This application claims priority to U.S. Provisional Patent Application Ser. No. 62/422,785 filed Nov. 16, 2016, the entire contents of which are incorporated herein by reference.

### BACKGROUND INFORMATION

In certain instances it can be desirable to clean a surface of a medical instrument while the surface is in vivo. For example, during laparoscopic surgery the vision through the laparoscope may be impaired. For example, the scope may become fogged, or the scope may be smeared by blood or other bodily fluids or tissues (e.g. interstitial fluid or fat tissue).

Currently, two different scope cleaning methods are commonly utilized. One method is to remove the laparoscope from the body, wipe the lens with a cloth, and reinsert it into the body. This method, though effective, is time consuming and causes the surgeon to lose visual of the surgical site, which can be considered dangerous, as surgical instruments typically remain inside the body.

The action of cleaning the laparoscope increases the length of time each surgical procedure takes, as well as decreases the amount of operating room (OR) time available to the hospital. Additionally, as patients undergo longer procedures, their time spent under anesthesia increases. As increased time under anesthesia has been shown to correlate to a rise in surgical complication rates and post-surgical infection rates, this excess time is not only wasteful, but also potentially medically and financially costly.

The other method is to wipe the laparoscope lens upon a nearby organ or tissue. While the laparoscope remains inside the body and takes less time to clean, this method is not often effective. When using either method, the surgeon must spend time relocating the surgical site within the body. The entire process is a hindrance and an annoyance for surgeons at minimum. Also, it is costly for hospitals, patients, and insurance companies due to wasted time, and possibly surgical complications and post-surgical infections.

There is presently a shortage of methods and devices that provide for effective devices and methods to clean a surface of a medical instrument in vivo. Exemplary embodiments of the present disclosure address these shortcomings.

### SUMMARY

Exemplary embodiments of the present disclosure allow for rapid and easy cleaning of an instrument (including for example a laparoscope) in vivo, negating the need for instrument removal from the body.

Embodiments of the present disclosure allow for laparoscope or other medical instrument users to clean a surface of the instrument (e.g. a lens) in vivo without having to remove the instrument from the body. It therefore significantly decreases the time it takes to clean the instrument surface as compared to current technologies, relieving a vast amount of the medical and financial pains placed on hospitals, clinicians, patients, and third-party payers.

Exemplary methods of cleaning an instrument include mechanical, optical, and pneumatic applications to clear unwanted objects, contaminants, particles, etc. from lens of the instrument.

Certain embodiments utilize a longitudinal member and a transverse member at the distal (lens) end of the laparoscope to be moved (e.g. swept, dragged, rotated, etc.) across the surface of the lens. In such embodiments, the transverse member may or may not be soaked or coated in a solution that assists in the removal of unwanted matter (e.g. objects, contaminants, particles, etc.) from the lens surface of the laparoscope. In certain embodiments, the solution may or may not be a saline solution, or a surfactant solution that appropriately and adequately removes bodily fluids and tissues from the lens surface, including but not limited to condensation, blood, interstitial fluid, fat tissue, etc.

Certain embodiments may implement surface cleaning materials, including one or multiple types of transverse members, such as flexible and/or absorbent components such as bristles, wipers, or sponges, with a longitudinal member configured as an elongated tube, rod, bar, or sheet. In some embodiments, the tube or rod fits around the laparoscope and inside a trocar. The distal end of the tube or rod can hold or house the transverse member (or members) that act as cleaning components. The scope can then be positioned (i.e. the scope retracted back or the tube or rod pushed forward) until it is a specific distance above the aforementioned cleaning components. The scope can then be pushed into and past the transverse member(s), which can perform a majority of the cleaning, at effective forces due to the stiffness and/or material properties of the transverse member(s), and the total combined surface area coverage.

In certain embodiments, an absorbent or sponge material may be implemented at the distal end of the longitudinal member to absorb or clean any remaining droplets or particles of elements that were not removed via the transverse member, if necessary. Exemplary embodiments of the device can clean different angled scopes of similar diameters with comparable efficacy.

Exemplary embodiments of the present disclosure comprise cleaning mechanisms and/or combinations of mechanisms with unique geometric, material and/or orientation specifications that provide the ability to effectively remove matter from a medical instrument surface in vivo.

In particular embodiments, a single configuration of the device can clean multiple styles of laparoscopes (e.g. different angles such as flat scopes, 30 degree scopes, 45 degree scopes and 70 degree scopes) at a relatively equivalent efficiency.

Exemplary embodiments are also compatible with current common laparoscope-trocar pairings, in contrast to typical existing systems. For example, a 5 mm laparoscope (approximately 5.5 mm diameter) can fit within a 5 mm trocar port (approximately 7.1 mm diameter). Current systems can require a larger port, while exemplary embodiments of the device disclosed herein can be made to fit within these tolerances.

Certain embodiments utilize one or more geometrical rubber/foam mats/wipers fixed to the distal end of the lens. As a scope is retracted into the sheath, the rubber wiper comes to rest in a passive position. As the scope is then pushed forward into the rubber wiper, the geometry and material of the wiper, in addition to its positioning relative to the scope surface allows the wiper to “scrape”, slide, or drag across the surface of the lens, comparable to that of a windshield wiper.

Certain embodiments utilize high frequency vibration either onto the laparoscope directly, or onto a mechanical component that in turn vibrates the laparoscope at a high frequency. Particular embodiments utilize optical intensity, frequency, continuous and/or pulsed light methods to remove debris from the lens. These light parameters can be altered via an attachment, or a built-in system.

Certain exemplary embodiments comprise a sheath that fits around a laparoscope or other type of cylindrical or tubular device that might require cleaning at a distal end (i.e. an endoscope). Particular embodiments include a transverse member that functions as a cleaning mechanism at the distal end of the device, near the lens or optical surface. In specific embodiments, the cleaning mechanism may comprise a hook-like, claw-like, broom-like, squeegee-like, or scraper-like geometry or configuration. During operation of exemplary embodiments, a component at a distal end of the transverse member is drawn across the surface, thereby cleaning debris from the surface.

In particular embodiments, the transverse member may be formed from a material comprising (or include a coating comprising) rubber, foam, plastic, or cloth material that does not scratch, harm, or impede the surface to be cleaned. In certain embodiments, the transverse member may include bristles, wipers, or an absorbent material (e.g. a material that is foam or sponge-like in nature). In specific embodiments, the transverse member may include a particular surface texture, including for example a surface finish of 0.01 microns-1000 microns, or more particularly 0.1-100 microns, or more particularly 1.0-10 microns.

In specific embodiments, the transverse member may include a surface porosity of 0-75 percent porosity, or more particularly 10-50 percent porosity, or more particularly 20-35 percent porosity.

In specific embodiments, the transverse member may include a particular rigidity, resilience, and/or flexibility to promote effective matter removal from the surface of the medical instrument. In certain embodiments, the material of the transverse member may have an elastic modulus of 0.005-5 gigapascals (GPa), or more particularly 0.05-2.0 GPa, or more particularly 0.5-1.5 GPa.

In some embodiments, the distal end of the transverse member may comprise a particular geometry of the cleaning edge (e.g., sharpened, rounded, multi-pronged, etc). In certain embodiments, the transverse member may include particular component angles and radii of approach and implementation where the transverse member is coupled to the longitudinal member (which may be configured as a sheath). For example, the angle between the transverse member proximal end and the longitudinal member may be between 0-90 degrees, or more particularly between 15 and 60 degrees, or more particularly between 30 and 45 degrees. In addition, the angle between the transverse member distal end and the surface to be cleaned may be between 0-180 degrees, or more particularly between 15 and 135 degrees, or more particularly between 30 and 90 degrees or more particularly between 45 and 60 degrees.

In addition, the transverse member can be configured so that it is able to remain out of view of the medical instrument, including adjacent to or near the medical instrument, as the user desires.

During operation of the device, it can expel dirty material from the lens surface and/or cleaning surface of the transverse member. Certain embodiments may also include an additional mechanism with the ability to convert stored potential energy to kinetic energy, including for example, a vibrational or “flick” mechanism. In certain embodiments, such a mechanism could be activated after transverse member initially moves across the surface of the medical instrument.

In certain laparoscopic embodiments, the device can be configured such that it is compatible with current commonly paired apparatus (e.g. a 5 mm diameter laparoscope with a commonly paired trocar). In certain embodiments, the device may be used in conjunction with a medical instrument having a shaft with a diameter of approximately 2.7 mm-approximately 12.0 mm and a trocar having a diameter of approximately 3.0 mm-13.0 mm.

In particular embodiments, the device may include a transverse member that is curved and has a radius of curvature of 1.3 mm-12.5 mm, or more particularly 2.0 mm-10.0 mm, or more particularly 3.0 mm-9.0 mm, or more particularly 4.0 mm-8.0 mm, or more particularly 5.0 mm to 7.0 mm. In specific embodiments, the device may include a longitudinal member that is tubular and had a diameter between approximately 3.0 mm and approximately 13.0 mm.

In certain embodiments, the device may comprise a constraint that aligns the transverse member appropriately with the surface of the medical instrument to be cleaned. This can be particularly important for different angled lenses or scopes. Exemplary embodiments can be actuated easily and quickly by manual or automated means, potentially via human input, robotic or mechanical input, or pneumatic input.

Exemplary embodiments include a device configured to clean a lens of a medical instrument in vivo. In certain embodiments, the device comprises a longitudinal member comprising a proximal end and a distal end, and a flexible transverse member comprising a proximal end and a distal end, where the proximal end of the flexible transverse member is coupled to the distal end of the longitudinal member. In particular embodiments, the distal end of the flexible transverse member is spaced apart or biased away from the proximal end of the transverse member, and the flexible transverse member is configured such that the distal end of the flexible transverse moves away from the longitudinal member (and/or moves away from the proximal end of the flexible transverse member) when a surface at an angle to the longitudinal member exerts a force on the distal end of the flexible transverse member in a direction parallel to the longitudinal member.

In some embodiments, the distal end of the flexible transverse member is configured to remove matter from the surface when the distal end of the flexible transverse moves away from the longitudinal member. In specific embodiments, the matter includes liquid matter (including for example, viscous fluids), or solid matter, or both liquid and solid matter. In certain embodiments, the surface is generally perpendicular to the longitudinal member, and in particular embodiments the surface is at an angle of approximately up to seventy degrees from the longitudinal member. In some embodiments, the longitudinal member is a tubular member, and in specific embodiments the tubular member has a diameter of between approximately 3.0 mm-and approximately 13.0 mm. In certain embodiments, the longitudinal member is a planar member. In particular embodiments, the longitudinal member and the flexible transverse member are formed from a unitary component, and in some embodiments, the longitudinal member and the flexible transverse member are separate components.

In specific embodiments, the flexible transverse member is curved or planar, and in certain embodiments has a radius of curvature of between approximately 1.3 mm-and approximately 12.5 mm. In particular embodiments, the flexible transverse member is formed from a plastic material, and in some embodiments the flexible transverse member comprises a deformable material coating, including for example, rubber, foam, fabric, or Velcro®. In some embodiments, the flexible transverse member comprises an extension member, and in specific embodiments the extension member is coupled to the distal end of the flexible transverse member. In certain embodiments, the extension member s angled toward the longitudinal member, and in particular embodiments, the flexible transverse member is a tubular member.

Specific embodiments include a system for cleaning a lens of a medical instrument in vivo, where the system comprises: a longitudinal member comprising a proximal end and a distal end; a transverse member coupled to the distal end of the longitudinal member; and a medical instrument comprising a shaft and a lens at a distal end of the shaft. In certain embodiments, the longitudinal member is configured to extend along the shaft of the medical instrument; the transverse member is biased toward the shaft of the medical instrument when the medical instrument is positioned in a first position such that a first distance between the lens and the proximal end is greater than a second distance between the transverse member and the proximal end; and the transverse member is configured to extend across the lens when the medical instrument is positioned in a second position such that the first distance between the lens and the proximal end is equivalent to the second distance between the transverse member and the proximal end.

In certain embodiments of the system, the distal end of the transverse member translates across the lens while maintaining contact with the lens as the longitudinal member is retracted back toward the proximal end of the medical instrument (or as the medical instrument is advanced relative to the longitudinal member). In particular embodiments, the transverse member is configured to retract across the lens when the medical instrument is moved from the second position to a third position such that a third distance between the lens and the proximal end is greater than the second distance between the transverse member and the proximal end. In some embodiments, the transverse member is configured to remove matter from the lens when the transverse member retracts across the lens. In specific embodiments, the matter includes liquid matter (including for example, viscous fluids), or solid matter or both liquid and solid matter. In certain embodiments, the longitudinal member is a tubular member, and in particular embodiments, the longitudinal member is a planar member.

In particular embodiments of the system, the longitudinal member and the transverse member are formed from a unitary component, and in some embodiments the longitudinal member and the transverse member are separate components. In some embodiments, the transverse member is curved or planar, and in specific embodiments the transverse member has a radius of curvature of between approximately 1.3 mm-and approximately 12.5 mm.

Certain embodiments include a device configured to clean a lens of a medical instrument in vivo, where the device comprises: a longitudinal member comprising a proximal end and a distal end; a first flexible transverse member: and a second flexible transverse member. In particular embodiments, the longitudinal member is a tubular member; the first flexible transverse member extends across the distal end of the longitudinal member; the second flexible transverse member extends across the distal end of the longitudinal member; and the first and second flexible members are configured to move across a surface at an angle to the longitudinal member when the surface moves past the distal end of the longitudinal member. In some embodiments, the surface is a lens of a medical instrument, and in specific embodiments the first flexible member is parallel to the second flexible member and the first flexible member is spaced apart from the second flexible member.

Exemplary embodiments include a method of cleaning a surface of a medical instrument in vivo, where the method comprises positioning a device adjacent to the medical instrument in vivo, where the device comprises a longitudinal member and a flexible transverse member, the flexible transverse member comprises a proximal end coupled to the longitudinal member, the flexible transverse member comprises a distal end, and the flexible transverse member is located adjacent the surface of the medical instrument. Exemplary embodiments of the method further comprise changing a relative position of the device and the medical instrument, where the distal end of the flexible transverse member engages the surface of the medical instrument, and the distal end of the flexible transverse member moves across the surface of the medical instrument.

In certain embodiments of the method, the medical instrument is a laparoscope, and in particular embodiments the surface of the medical instrument is a lens. In some embodiments, the distal end of flexible transverse member moves toward the proximal end of the flexible transverse member when the distal end of the flexible transverse member moves across the surface of the medical instrument. In specific embodiments, the distal end of the flexible transverse member removes matter from the surface of the medical instrument when the distal end of the flexible transverse moves across the surface of the medical instrument.

In certain embodiments of the method, the matter includes liquid matter (including for example, viscous fluids), or solid matter or both liquid and solid matter. In particular embodiments, the surface of the medical instrument is generally perpendicular to the longitudinal member. In some embodiments, the surface of the medical instrument is at an angle of approximately forty-five degrees from the longitudinal member. In specific embodiments, the longitudinal member is a tubular member, and in certain embodiments the tubular member has a diameter of between approximately 3.0 mm-and approximately 13.0 mm.

In particular embodiments of the method, the longitudinal member is a planar member. In some embodiments, the longitudinal member and the flexible transverse member are formed from a unitary component and in specific embodiments the longitudinal member and the flexible transverse member are separate components. In certain embodiments, the flexible transverse member is curved or planar, and in particular embodiments the flexible transverse member has a radius of curvature of between approximately 1.3 mm-and approximately 12.5 mm. In certain embodiments, the flexible transverse member is formed from a plastic material.

In particular embodiments, the flexible transverse member comprises a a coating is selected from the group consisting of rubber, foam and fabric.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “approximately, “about” or “substantially” mean, in general, the stated value plus or minus 10%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

FIG. 1 illustrates a schematic of a device according to a first exemplary embodiment of the present disclosure during use.

FIG. 2 illustrates a schematic of a device according to a second exemplary embodiment of the present disclosure during use.

FIG. 3 illustrates a schematic of a device according to a third exemplary embodiment of the present disclosure.

FIG. 4 illustrates a schematic of a device according to a fourth exemplary embodiment of the present disclosure.

FIG. 5 illustrates a schematic of a device according to a fifth exemplary embodiment of the present disclosure.

FIG. 6 illustrates a schematic of a device according to a sixth exemplary embodiment of the present disclosure.

FIG. 7 illustrates a schematic of a device according to a seventh exemplary embodiment of the present disclosure.

FIG. 8 illustrates a schematic of a device according to an eighth exemplary embodiment of the present disclosure.

### DETAILED DESCRIPTION OF ILLUSTRATIVE EMBODIMENTS

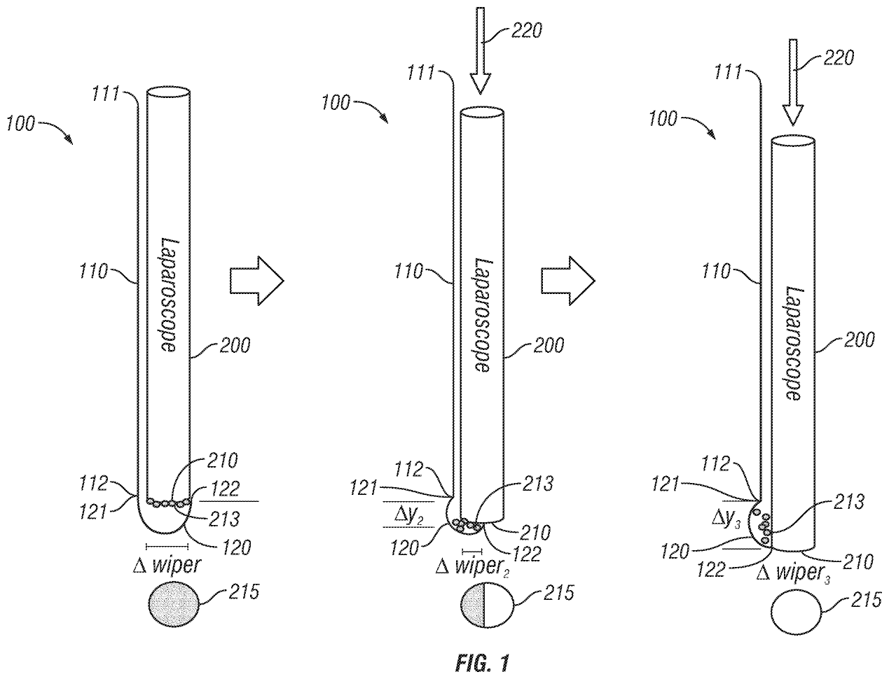

Referring now to FIG. 1, an exemplary embodiment of a device 100 is shown during use cleaning a medical instrument 200. In the embodiment shown, device 100 is configured to clean a surface 210 (e.g. a lens) of medical instrument 200 in vivo. In this embodiment, device 100 comprises a longitudinal member 110 comprising a proximal end 111 and a distal end 112 Device 100 also comprises a flexible transverse member 120 comprising a proximal end 121 and a distal end 122, where proximal end 121 of flexible transverse member 120 is coupled to distal end 112 of longitudinal member 110. As used herein, distal end 112 of longitudinal member 110 comprises a region of longitudinal member 110 that is distal from proximal end 112. Accordingly, a portion (e.g. 5, 10 or 20 percent) of longitudinal member 110 may extend beyond the coupling point of flexible transverse member 120 and longitudinal member 110 (e.g. may extend away from proximal end 111).

In certain embodiments, longitudinal member 110 and flexible transverse member 120 may be formed from a unitary component, while in other embodiments, longitudinal member 110 and flexible transverse member 120 may be separate components.

In FIG. 1, device 100 is shown during use with progression of steps from left to right.

In the far left view, device 100 is shown in its unaltered position (e.g. before surface 210 has moved distal end 122 of flexible transverse member 120). In the views moving to the right in FIG. 1, medical instrument 200 is moved relative to device 100 in the direction indicated by arrows 220. As medical instrument 200 is moved in this direction, surface 210 is moved in a direction parallel to longitudinal member 110 and away from distal end 111. Surface 210 exerts a force on distal end 122 of flexible transverse member 120 during the movement of medical instrument 200 relative to device 100.

When surface 210 exerts a force on distal end 122, transverse member 120 is configured such that distal end 122 moves away from proximal end 121 of flexible transverse member 120. In exemplary embodiments, distal end 122 is configured to remove matter 213 from surface 210 when distal end 122 moves across surface 210 toward proximal end 121 of transverse member 120.

When surface 210 is a lens (e.g. and medical instrument 200 is a laparoscope) the removal of matter 213 from surface 210 can allow a user to more clearly view a subject area being examined by medical instrument 200. A simulated view 215 through medical instrument 200 is shown below device 100 and medical instrument 200 in FIG. 1. As shown in the progression of steps from left to right, as matter 213 is removed from surface 210, the view through medical instrument 200 progresses from an obscured view to a clean view (e.g. from a darker view to a lighter view).

In certain embodiments, device 100 can be configured to remove matter 213 from surface 210 of medical instrument 200 in vivo. For example, in certain embodiments, longitudinal member 110 may be configured as a tubular member such that a shaft of medical instrument 200 is disposed within the tubular member. In specific embodiments, longitudinal member 110 may be a tubular member configured to be disposed within a trocar, such that longitudinal member 110 is located between the trocar and medical instrument 200.

The embodiment of FIG. 1 illustrates medical instrument 200 moving in a direction of arrow 220. However, it is understood that device 100 may instead be moved in a direction opposite of arrow 220. Device 100 is configured to remove matter 213 from surface 210 when the relative position between device 100 and medical instrument 200 is changed while distal end 122 is engaged with surface 210 (such that the distance between proximal end 111 and surface 210 is increased). Accordingly, device 100 is configured to clean surface 210 whether device 100 is held stationary and medical instrument 200 is moved, or device 100 is moved and medical instrument 200 is held stationary, or if both device 100 and medical instrument 200 are moved.

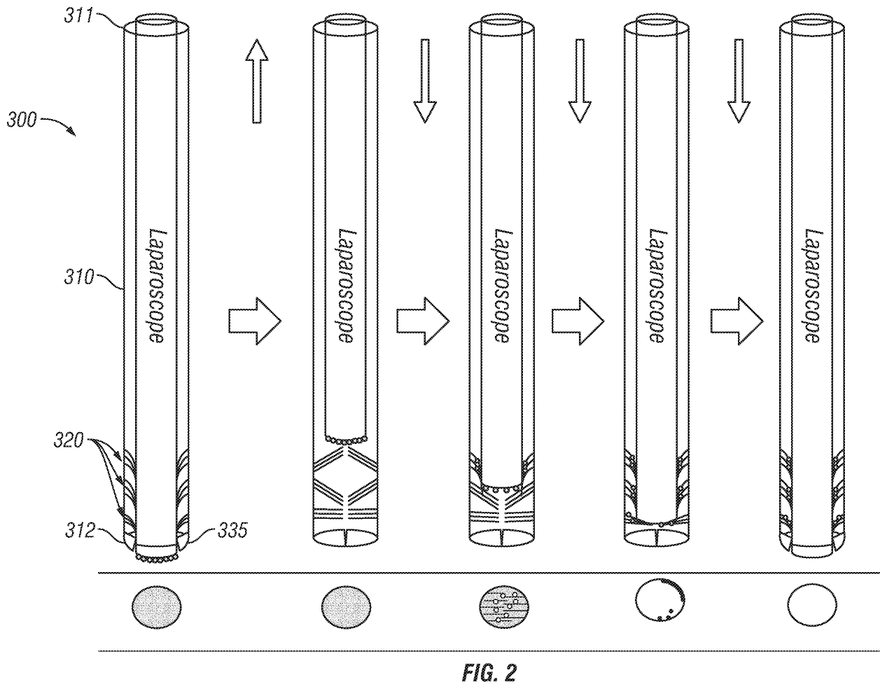

Referring now to FIG. 2, a second embodiment comprises a device 300 for cleaning a surface of a medical instrument in vivo. In FIG. 2, reference numbers for certain features equivalent to those of the embodiment in FIG. 1 will not be repeated. This embodiment is similar to the previously-described embodiment of FIG. 1, but includes a longitudinal member 310 and multiple transverse members 320 and 335. In certain embodiments, transverse members 320 may be configured as bristles or fibers. In addition, device 300 may also comprise transverse members 335 configured as one or more sponges at distal end 312 of longitudinal member 310. In certain embodiments, sponge 335 may be split to allow the medical instrument to pass through sponge 335. During operation, device 300 operates in a manner similar to device 100 to clean matter from the end surface (e.g. lens) of the medical instrument.

In certain embodiments, transverse members 320 may be configured as bristles or fibers. In addition, device 300 may also comprise a sponge 335 at distal end 312 of longitudinal member 310. In certain embodiments, sponge 335 may be split to allow the medical instrument to pass through sponge 335. During operation, device 300 operates in a manner similar to device 100 to clean matter from the end surface (e.g. lens) of the medical instrument.

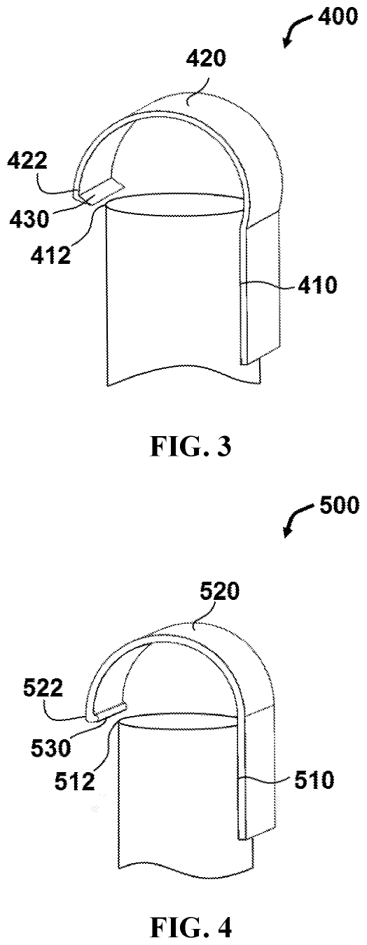

Referring now to FIG. 3, another embodiment comprises a device 400 fir cleaning a surface of a medical instrument in vivo. In FIG. 3 and the subsequent figures, reference numbers for certain features equivalent to those of the embodiment in FIG. 1 may not be repeated. This embodiment is also similar to the previously-described embodiment of FIG. 1, but includes a longitudinal member 410 and a transverse member 420 with an extension member 430. In this embodiment, extension member 430 is coupled to distal end 422 of transverse member 420. It is understood that extension member 430 may be coupled to other portions of transverse member 420 near distal end 422 and need not be coupled directly to distal end 422. In this embodiment, extension member 430 is angled toward longitudinal member 410. In the illustrated embodiment, extension member 430 is configured with a profile with a distinct edge angled toward longitudinal member. During use of device 400, extension member 430 can assist in removing matter from a surface (e.g. a lens) of a medical instrument as the surface moves past a distal end 412 of longitudinal member 410. In some embodiments extension member 430 may be formed from the same material as transverse member 420 (e.g. rubber, plastic, or other suitable material) and may be integral with transverse member 420. In other embodiments, extension member 430 may be a separate component from transverse member 420 and may be formed from a different material than transverse member 420.

Referring now to FIG. 4, another embodiment comprises a device 500 for cleaning a surface of a medical instrument in vivo. This embodiment is similar to the previously described embodiment of FIG. 3, but includes a longitudinal member 510 and a transverse member 520 with an extension member 530 having a curved or rounded profile. In this embodiment, extension member 530 is coupled to distal end 522 of transverse member 520. It is understood that extension member 530 may be coupled to other portions of transverse member 520 near distal end 522 and need not be coupled directly to distal end 522. Similar to the previous embodiment, extension member 530 can assist in removing matter from a surface (e.g. a lens) of a medical instrument as the surface moves past a distal end 512 of longitudinal member 510 during use, In some embodiments extension member 530 may be formed from the same material as transverse member 520 (e.g. rubber, plastic, or other suitable material) and may be integral with transverse member 520. In other embodiments, extension member 530 may be a separate component from transverse member 520 and may be formed from a different material than transverse member 520.

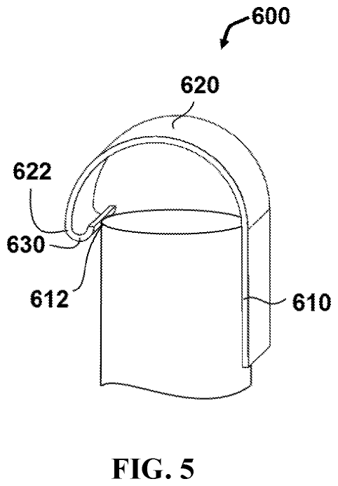

Referring now to Referring now to FIG. 5, another embodiment comprises a device 600 for cleaning a surface of a medical instrument in vivo. This embodiment is similar to the previously-described embodiment of FIG. 4, but includes a longitudinal member 610 (with distal end 612) and a transverse member 620 (with distal end 622) with an extension member 630 having a curved profile rather than a distinct edge. Similar to the previous embodiment, extension member 630 can assist in removing matter from a surface of a medical instrument during use. In some embodiments extension member 630 may be formed from the same material as transverse member 620 (e.g. rubber, plastic, or other suitable material) and may be integral with transverse member 620. In other embodiments, extension member 630 may be a separate component from transverse member 620 and may be formed from a different material than transverse member 620.

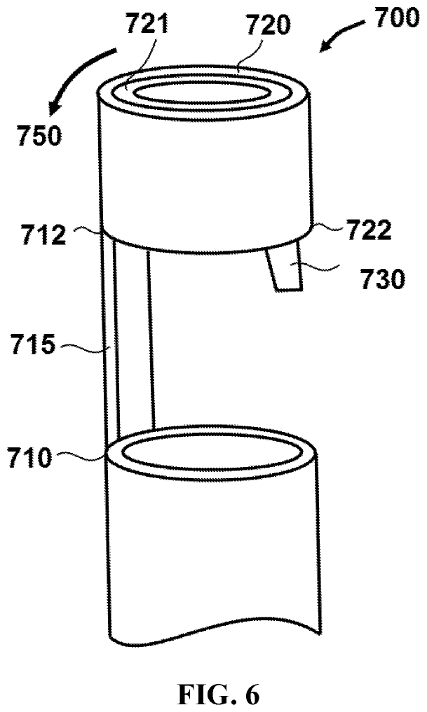

Referring now to FIG. 6, another embodiment comprises a device 700 for cleaning a surface of a medical instrument in vivo. This embodiment is similar to the previously-described embodiments, but includes a longitudinal member 710 and a transverse member 720. In this embodiment, longitudinal member 710 may comprise a tubular shape with an intermediate flexible portion 715 that allows transverse member 720 to flex in the direction of arrow 750. In the embodiment shown, transverse member 720 comprises an extension member 730 that can assist in removing matter from a surface of a medical instrument during use.

In the illustrated embodiment, transverse member 720 comprises an outer portion 722 and an inner portion 721. In a particular embodiment, outer portion 722, flexible portion 715 and longitudinal member 710 may be formed by removing material from a unitary tubular member. For example, material from a unitary tubular member can be removed in the region adjacent flexible portion 715. In certain embodiments, inner portion 721 and extension member 730 may also be formed from a unitary material, including for example, flexible tubing. Similar to the previous embodiment, extension member 730 can assist in removing matter from surface of a medical instrument during use. In certain embodiments, transverse member 720 may also be a unitary member that dos not comprise separate outer and inner portions.

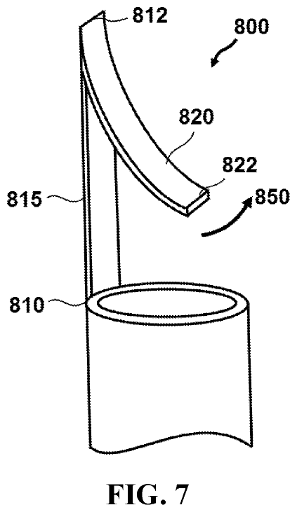

Referring now to FIG. 7, another embodiment comprises a device 800 for cleaning a surface of a medical instrument in vivo. This embodiment includes a longitudinal member 810, an intermediate member 815 and a transverse member 820. In this embodiment, transverse member 820 is coupled to intermediate member at a first end 812, and a second end 822 can flex in the direction of arrow 850 during use. Transverse member 820 may be formed from any suitable flexible material (including for example silicone) that can assist in removing matter from a surface of a medical instrument during use. For purposes of clarity, second end 822 is shown spaced apart from intermediate member 815 in FIG. 7. It is understood that transverse member 820 may be configured such that second end 822 is located closer to intermediate member 815 when device 800 is not in use (i.e. transverse member 820 may be substantially parallel to intermediate member 815 when not in use).

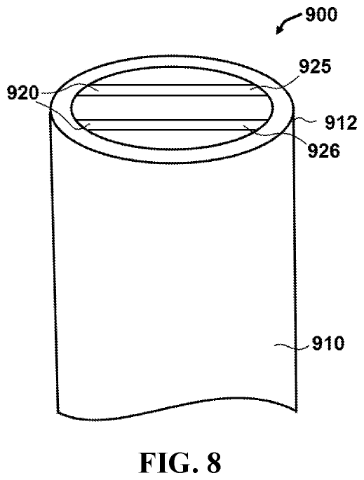

Referring now to FIG. 8, another embodiment comprises a device 900 for cleaning a surface of a medical instrument in vivo. This embodiment includes a longitudinal member 910 configured as a tubular member. In addition, device 900 comprises a transverse member 920 coupled to a distal end 912 of longitudinal member 910. In this embodiment, transverse member 920 comprises a first flexible member 925 and a second flexible member 926 that extend across distal end 912. First and second flexible members 925 and 926 are configured to move across a surface at an angle to the longitudinal member when the surface moves past the distal end of the longitudinal member. Accordingly, first and second flexible members 925 and 926 can assist in removing matter from a surface of a medical instrument during use.

In certain embodiments, first and second flexible members 925 and 926 can be spaced apart such that the surface passes between flexible members. It is understood that the spacing first and second flexible members 925 and 926 shown in FIG. 8 is merely exemplary for illustration purposes. In certain embodiments, first and second flexible members 925 and 926 may be adjacent such that both flexible members pass to one side of the surface as the surface extends past distal end 912. In particular embodiments, first and second flexible members 925 and 926 may be formed from a unitary loop of material (e.g. rubber or other suitable material) that is coupled to distal end 912.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:

U.S. Pat. No. 5,392,766

U.S. Pat. No. 5,658,273

U.S. Pat. No. 6,354,992

U.S. Pat. No. 8,001,984

U.S. Pat. No. 8,047,215

U.S. Pat. No. 8,535,220

U.S. Pat. No. 8,690,764

U.S. 20020065450

U.S. 20060293559

U.S. 20100139018

U.S. 20120101338

U.S. 20140171739

CA 2400381

CN 102578999

CN 203917219

CN 202892095

1. A device configured to clean a surface of a medical instrument in vivo, the device comprising:
a longitudinal member comprising a proximal end and a distal end; and
a flexible transverse member comprising a proximal end and a distal end, wherein:
the proximal end of the flexible transverse member is coupled to the distal end of the longitudinal member;
the distal end of the flexible transverse member is spaced apart from the proximal end of the transverse member; and
the flexible transverse member is configured such that the distal end of the flexible transverse member moves away from the longitudinal member when a surface at an angle to the longitudinal member exerts a force on the distal end of the flexible transverse member in a direction parallel to the longitudinal member.

2. The device of claim 1 wherein the distal end of the flexible transverse member is configured to remove matter from the surface when the distal end of the flexible transverse moves away from the longitudinal member.

3. The device of claim 2 wherein the matter includes liquid matter.

4. The device of claim 2 wherein the matter includes solid matter.

5. The device of claim 2 wherein the matter includes viscous fluid.

6. The device of claim 1 wherein the surface is generally perpendicular to the longitudinal member.

7. The device of claim 1 wherein the surface is at an angle of approximately seventy degrees from the longitudinal member.

8. The device of claim 1 wherein the longitudinal member is a tubular member.

9. The device of claim 8 wherein the tubular member has a diameter of between approximately 3.0 mm-and approximately 13.0 mm.

10. The device of claim 1 wherein the longitudinal member is a planar member.

11. The device of claim 1 wherein the longitudinal member and the flexible transverse member are formed from a unitary component.

12. The device of claim 1 wherein the longitudinal member and the flexible transverse member are separate components.

13. The device of claim 1 wherein the flexible transverse member is curved or planar.

14. The device of claim 1 wherein the flexible transverse member has a radius of curvature of between approximately 1.3 mm-and approximately 12.5 mm.

15. The device of claim 1 wherein the flexible transverse member is formed from a plastic material.

16. The device of claim 1 wherein the flexible transverse member comprises a deformable material coating.

17. The device of claim 16 wherein the deformable material coating is selected from the group consisting of rubber, foam, and fabric.

18. The device of claim 1 wherein the flexible transverse member comprises an extension member.

19. The device of claim 18 wherein the extension member is coupled to the distal end of the flexible transverse member.

20. The device of claim 18 wherein the extension member is angled toward the longitudinal member.

21. The device of claim 18 wherein the flexible transverse member is a tubular member.

22. A system for cleaning a lens of a medical instrument in vivo, the system comprising:
a longitudinal member comprising a proximal end and a distal end;
a transverse member coupled to the distal end of the longitudinal member; and
a medical instrument comprising a shaft and a lens at a distal end of the shaft, wherein:
the longitudinal member is configured to extend along the shaft of the medical instrument;
the transverse member is biased toward the shaft of the medical instrument when the medical instrument is positioned in a first position such that a first distance between the lens and the proximal end is greater than a second distance between the transverse member and the proximal end; and
the transverse member is configured to extend across the lens when the medical instrument is positioned in a second position such that the first distance between the lens and the proximal end is equivalent to the second distance between the transverse member and the proximal end.

23. The system of claim 22 wherein the distal end of the transverse member translates across the lens while maintaining contact with the lens as the longitudinal member is retracted back toward the proximal end of the medical instrument.

24. The system of claim 22 wherein the transverse member is configured to retract across the lens when the medical instrument is moved from the second position to a third position such that a third distance between the lens and the proximal end is greater than the second distance between the transverse member and the proximal end.

25. The system of claim 24 wherein the transverse member is configured to remove matter from the lens when the transverse member retracts across the lens.

26. The system of claim 25 wherein the matter includes liquid matter.

27. The system of claim 25 wherein the matter includes solid matter.

28. The system of claim 25 wherein the matter includes viscous fluid.

29. The system of claim 22 wherein the longitudinal member is a tubular member.

30. The system of claim 22 wherein the longitudinal member is a planar member.

31. The system of claim 22 wherein the longitudinal member and the transverse member are formed from a unitary component.

32. The system of claim 22 wherein the longitudinal member and the transverse member are separate components.

33. The system of claim 22 wherein the transverse member is curved or planar.

34. The system of claim 33 wherein the transverse member has a radius of curvature of between approximately 1.3 mm-and approximately 12.5 mm.

35. A device configured to clean a lens of a medical instrument in vivo, the device comprising:
a longitudinal member comprising a proximal end and a distal end;
a first flexible transverse member; and
a second flexible transverse member, wherein:
the longitudinal member is a tubular member;
the first flexible transverse member extends across the distal end of the longitudinal member;
the second flexible transverse member extends across the distal end of the longitudinal member; and
the first and second flexible members are configured to move across a surface at an angle to the longitudinal member when the surface moves past the distal end of the longitudinal member.

36. The device of claim 35 wherein the surface is a lens of a medical instrument.

37. The device of claim 36 wherein the first flexible member is parallel to the second flexible member and the first flexible member is spaced apart from the second flexible member.

38. A method of cleaning a surface of a medical instrument in vivo, the method comprising:
positioning a device adjacent to the medical instrument in vivo, wherein:
the device comprises a longitudinal member and a flexible transverse member;
the flexible transverse member comprises a proximal end coupled to the longitudinal member;
the flexible transverse member comprises a distal end; and
the flexible transverse member is located adjacent the surface of the medical instrument;
changing a relative position of the device and the medical instrument, wherein:
the distal end of the flexible transverse member engages the surface of the medical instrument; and
the distal end of the flexible transverse member moves across the surface of the medical instrument.

39. The method of claim 38 wherein the medical instrument is a laparoscope.

40. The method of claim 38 wherein the surface of the medical instrument is a lens.

41. The method of claim 38 wherein the distal end of flexible transverse member moves away from the longitudinal member when the distal end of the flexible transverse member moves across the surface of the medical instrument.

42. The method of claim 38 wherein the distal end of the flexible transverse member removes matter from the surface of the medical instrument when the distal end of the flexible transverse moves across the surface of the medical instrument.

43. The method of claim 42 wherein the matter includes liquid matter.

44. The method of claim 42 wherein the matter includes solid mailer.

45. The method of claim 42 wherein the matter includes viscous fluid.

46. The method of claim 38 wherein the surface of the medical instrument is generally perpendicular to the longitudinal member.

47. The method of claim 38 wherein the surface of the medical instrument is at an angle of approximately seventy degrees from the longitudinal member.

48. The method of claim 38 wherein the longitudinal member is a tubular member.

49. The method of claim 48 wherein the tubular member has a diameter of between approximately 3.0 mm-and approximately 13.0 mm.

50. The method of claim 38 wherein the longitudinal member is a planar member.

51. The method of claim 38 wherein the longitudinal member and the flexible transverse member are formed from a unitary component.

52. The method of claim 38 wherein the longitudinal member and the flexible transverse member are separate components.

53. The method of claim 38 wherein the flexible transverse member is curved or planar.

54. The method of claim 38 wherein the flexible transverse member has a radius of curvature of between approximately 1.3 mm-and approximately 12.5 mm.

55. The method of claim 38 wherein the flexible transverse member is formed from a plastic material.

56. The method of claim 38 wherein the flexible transverse member comprises a coating is selected from the group consisting of rubber, foam and fabric.
